(19) United States
(12) Patent Application Publication
(10) Pub. No.: US 2010/0144622 A1
(43) Pub. Date: Jun. 10, 2010

(54) METHODS FOR PRODUCTION OF RECOMBINANT PLASMINOGEN AND PLASMIN POLYPEPTIDES

(76) Inventors: Xinli Lin, Gaitersburg, MD (US); Daniel Medynski, Oakland, CA (US)

Correspondence Address: MORRISON & FOERSTER LLP, 755 PAGE MILL RD, PALO ALTO, CA, 94304-1018, US

(21) Appl. No.: 12/443,075

(22) PCT Filed: Sep. 28, 2007
(86) PCT No.: PCT/US07/21061

§ 371 Date: Feb. 11, 2010

Related U.S. Application Data
(60) Provisional application No. 60/848,456, filed on Sep. 29, 2006.

(51) Int. Cl. A61K 38/17; C07K 14/435; C07K 1/22; C07K 1/18; C07K 1/16; A61P 7/02

(52) U.S. Cl. 514/12; 530/394; 530/413; 530/416; 530/417

(57) ABSTRACT

Methods of producing properly refolded recombinant plasminogen and plasmin polypeptide are provided. Denatured recombinant plasminogen polypeptide is refolded by first solubilizing the polypeptide with a chaotroph and reducing and oxidizing agents at high pH, followed by refolding in the presence of reduced concentration of chaotroph and reducing and oxidizing agents and in the presence of arginine.

### CROSS-REFERENCE TO RELATED APPLICATIONS

This application claims the priority benefit of provisional patent applications U.S. Ser. No. 60/848,456, filed Sep. 29, 2006, which is incorporated in its entirety by reference.

### STATEMENT REGARDING FEDERALLY SPONSORED RESEARCH OR DEVELOPMENT

Not applicable.

### FIELD OF THE INVENTION

This invention relates to methods for production of recombinant plasminogen polypeptides.

### BACKGROUND OF THE INVENTION

Plasmin (Plm) is a serine protease with several important physiological roles, including dissolving blood clots. Plasmin is normally found circulating inertly in the blood in its zymogen form, plasminogen (Plg): a 791 amino acid single-chain glycoprotein (Mr˜87 KDa) that possesses the capacity to bind to newly formed blood clots. Its activation is effected by digestion of the peptide bond between arginine 561 and valine 562 (R^{561▾}V⁵⁶²) by tissue plasminogen activator (tPA) or urokinase (uPA) trapped in the blood clot. Cleavage at this peptide bond transforms the single chain protein into two separate subunits interconnected by two essential disulfide bridges. The A chain of the plasmin molecule consists of five triple-loop disulfide kringle (Kr) domains (approximately 78-80 amino acids each), while the B chain contains a “linker” region of 20 amino acids and a serine protease domain (approximately 228 amino acids). The newly formed plasmin actively digests the fibrin in the clot, thereby dissolving it.

Through laboratory manipulations, two des-kringle variants of plasminogen with potential pharmacological application, miniplasmin (miniPlm) and microplasmin (μPlm), were elucidated two decades ago. MiniPlm consists of only the kringle-5 domain, the linker, and the serine protease domain, while μPlm consists of only the linker and serine protease domain. MiniPlm is produced by digestion of full length plasmin with neutrophil elastase (1, 2), which cleaves specifically at the peptide bond between valine 441 and valine 442 (V^{441▾}V⁴⁴²). μPlm was initially produced by pH 11 base-mediated cleavage of full length plasmin at the peptide bond between arginine 530 and lysine 531 (R^{530▾}K⁵³¹) (3, 4). In the laboratory, μPlm and miniPlm can be generated from their zymogen precursors, microplasminogen (μPlg) and miniplasminogen (miniPlg) respectively, by cleavage at the same peptide bond as was described for plasminogen. Similarly, two separate subunits interconnected by the same two disulfide bridges are generated in each case. Removal of either one or both of these two disulfide bridges by mutagenesis renders the activated serine protease domain non-functional (5).

Recent advances in thrombolytic therapy have, in part, been driven by significant technical advances in catheter design and delivery, permitting local drug delivery directly into a clot. These innovations are particularly relevant to treatment of peripheral arterial occlusion disease (PAOD). In landmark studies, Novokhatny, Marder, and coworkers (6, 7) were able to show, in an animal model of abdominal aorta thrombosis and in in vitro models, that serum-derived plasmin was equivalent to recombinant tissue plasminogen activator (tPA) in dissolution of the clot during unimpeded blood flow. More importantly, plasmin was clearly superior under conditions of restricted blood flow where Plg substrate was not replenished. Furthermore, plasmin caused dramatically less bleeding at a hemostatically stable ear puncture site distant from the thrombus site. The advantage of plasmin was realized because of its remarkably short half-life relative to tPA (0.02 seconds vs.˜15 minutes in vivo) (8).

These studies “opened the door” to similar studies demonstrating the utility of μPlm for treatment of PAOD and ischemic stroke. Nagai and coworkers (8) compared local catheter mediated delivery of serum-derived plasmin, recombinant μPlm isolated from yeast P. pastoris, and recombinant tPA and showed similar efficacy in clot dissolution using a rabbit extracorporeal loop thrombolysis model to simulate PAOD and a mouse middle cerebral artery model to simulate ischemic stroke.

Plasmin is typically isolated from blood. Nagai and coworkers have produced μPlg recombinantly at high yield using P. pastoris. Nagai et al., J. Thromb. Haemost. 1:307-313, 2003. μPlg has also been produced utilizing a baculovirus expression system (5), but with relatively low yield. Wang et al., Protein Sci. 4:1768-1779, 1995. There remains a need to recombinantly produce biologically active plasmin at a pharmaceutical scale.

Methods for refolding proteins from E. coli inclusion bodies have been reported. See, e.g., U.S. Pat. No. 6,583,268; U.S. Pub. Nos. US 2003/070242; US 2003/0199676; US 2004/0265298; and US 2005/0227920; and PCT Pub. Nos. WO 03/039491; WO 2004/094344; WO 01/55174; WO 2005/05830. Methods disclosed in these references include a step of refolding proteins by reducing pH slowly from a high pH to a physiological pH.

All references, publications, and patent applications disclosed herein are hereby incorporated by reference in their entirety.

### BRIEF SUMMARY OF THE INVENTION

The invention provides methods for producing biologically active recombinant plasminogen polypeptides. These refolded polypeptides can be treated with plasminogen activator, such as urokinase to generate biologically active plasmin for pharmaceutical use.

The invention provides methods for refolding a recombinant plasminogen polypeptide, comprising: (a) solubilizing a plasminogen polypeptide in a solubilization buffer, said solubilization buffer comprising a high concentration of chaotroph, a reducing agent, redox reagents, and having a pH of about 9.0 to about 11.0, thereby producing a solubilized plasminogen polypeptide solution; and (b) rapidly diluting said solubilized plasminogen polypeptide solution with a refolding buffer by adding said solubilized plasminogen polypeptide solution into the refolding buffer, thereby producing diluted solubilized plasminogen polypeptide solution, wherein the refolding buffer comprises arginine and has a pH of about 8.0 to about 10.0; and (c) incubating the diluted solubilized plasminogen polypeptide solution, thereby producing a refolded plasminogen polypeptide. The plasminogen polypeptides can be expressed in bacterial cells, and the denatured polypeptides in the inclusion bodies can be refolded.

The invention also provides biologically active plasminogen polypeptides and plasmin polypeptides produced by methods described herein.

The invention also provides compositions comprising refolded plasminogen polypeptides produced from bacterial cells, such as E. coli.

The invention also provides compositions (including pharmaceutical compositions) comprising biologically active plasmin polypeptides produced from bacterial cells, such as E. coli.

### BRIEF DESCRIPTION OF THE DRAWINGS

FIG. 1 shows a diagram of plasminogen structure. Domains indicated are: PAP, preactivation peptide; A chain: kringles 1-5 (K1-K5); and B chain, catalytic domain.

FIG. 2A shows the SDS-PAGE analysis (with β-ME reduction) of whole cell extracts of cells containing expression plasmids for μPlg or miniPlg after induction with IPTG for 3 hrs. Lanes: Lane 1. MW std; Lane 2. μPlg−IPTG induction; Lane 3. μPlg+IPTG induction; Lane 4. miniPlg−IPTG induction; Lane 5. miniPlg+IPTG induction.

FIG. 2B shows the SDS-PAGE (with β-ME reduction) analysis of purified μPlg and miniPlg inclusion bodies. Lanes: Lane 1. MW std; Lane 2. μPlg; Lane 3. miniPlg.

FIG. 3A shows the amino acid sequence of authentic microplasmin as originally generated after pH 11 treatment by Wu et al. and the recombinant construct (r-hu-μPlg) used in the example. Wu et al., Proc. Natl. Acad. Sci. U.S.A. 84:8292-8295, 1987; Wu et al., Proc. Natl. Acad. Sci. U.S.A. 84:8793-8795, 1987. The numbering system is derived from full length plasmin. Arrow pointing downward indicates the authentic urokinase activation cleavage site R^{561∇}V⁵⁶², while the upward pointing arrow indicates an artificial urokinase cleavage site engineered to trim away the molecule of inauthentic amino acids in plasmid construct following activation. The inauthentic amino acids are underlined by a single line. Only an inauthentic valine will remain. Boxed cysteines indicate essential disulfide bridges between chain A and chain B after activation.

FIG. 3B shows the schematic diagram of recombinant miniplasmin construct used in the Example.

FIG. 4A shows the Superdex 75 chromatogram of refolded μPlg.

FIG. 4B shows the SDS-PAGE of μPlg fractions isolated in FIG. 4A. Lanes: Lane 1. MW std; Lane 2. F (fraction) 42 non-reduced; Lane 3. F42 reduced; Lane 4. F50 non-reduced; Lane 5. F50 reduced. Non-reduced, non-activated (full length) μPlg has an apparent mobility faster (Mr˜29 KDa) than the reduced form (Mr˜32 KDa) (lane 4 vs. lane 5). The lower molecular weight bands in the reduced lane 3 represent auto-activated, fragments as discussed in the Example.

FIG. 4C shows the Superdex 75 of refolded miniPlg. The elution peak on the left consists of multimeric unfolded forms of miniPlg while the protein peak on the right consists primarily of miniPlg monomers.

FIG. 4D shows the SDS PAGE of Superdex 75 chromatography in FIG. 4C. Lanes: Lane 1. MW std; Lane 2. non-reduced F28, Lane 3. reduced F28; Lane 4. non-reduced F45; Lane 5. reduced F45.

FIG. 4E shows a sample of purified miniPlg stored in alkaline pH buffer (20 mM Tris, pH 9.0, 0.2 M arginine, 0.15 M NaCl) auto-activated after storage at 4° C. for 1 week. Lanes: Lane 1. MW std; Lane 2. reduced sample of miniPlg.

FIG. 5A shows Hanes plot kinetic comparison of refolded μPlm, miniPlm, and commercially purchased plasmin using amidolytic chromogenic substrate S-2403.

FIG. 5B shows a summary of the kinetic parameters obtained in FIG. 5A.

### DETAILED DESCRIPTION OF THE INVENTION

The instant invention provides methods for the production of recombinant, biologically active plasminogen and plasmin polypeptides. The instant invention also provides compositions (including pharmaceutical compositions) comprising biologically active plasminogen and plasmin polypeptides.

The practice of the present invention will employ, unless otherwise indicated, conventional techniques of immunology, molecular biology, microbiology, cell biology and recombinant DNA, which are within the skill of the art. See, e.g., Molecular Cloning: a laboratory manual, 2^{nd} edition Sambrook, et al. (1989); Current Protocols In Molecular Biology F. M. Ausubel, et al. eds., (1987); the series Methods In Enzymology, Academic Press, Inc.; PCR 2: A Practical Approach, M. J. MacPherson, B. D. Hames and G. R. Taylor, eds. (1995), and Antibodies, A Laboratory Manual, Harlow and Lane, eds. (1988).

It should be noted that, as used herein, the singular form “a”, “an”, and “the” includes plural references unless indicated otherwise.

It is understood that aspect and embodiments of the invention described herein include “consisting” and/or “consisting essentially of” aspects and embodiments.

### Plasminogen and Plasmin Polypeptides

Plasminogen polypeptide includes any naturally occurring species (such as full length protein from any mammalian (e.g., human, farm animals, sport animals, pets, primates, horses, dogs, cats, mice and rats), biologically active polypeptide fragments, and variants (including naturally occurring and non-naturally occurring), including functionally equivalent variants which do not significantly affect their biological properties and variants which have enhanced or decreased activity. Examples of variants include one or more amino acid substitution (e.g., conservative substitution), one or more deletions or additions of amino acids which do not significantly change the folding or functional activity of the protein or polypeptide.

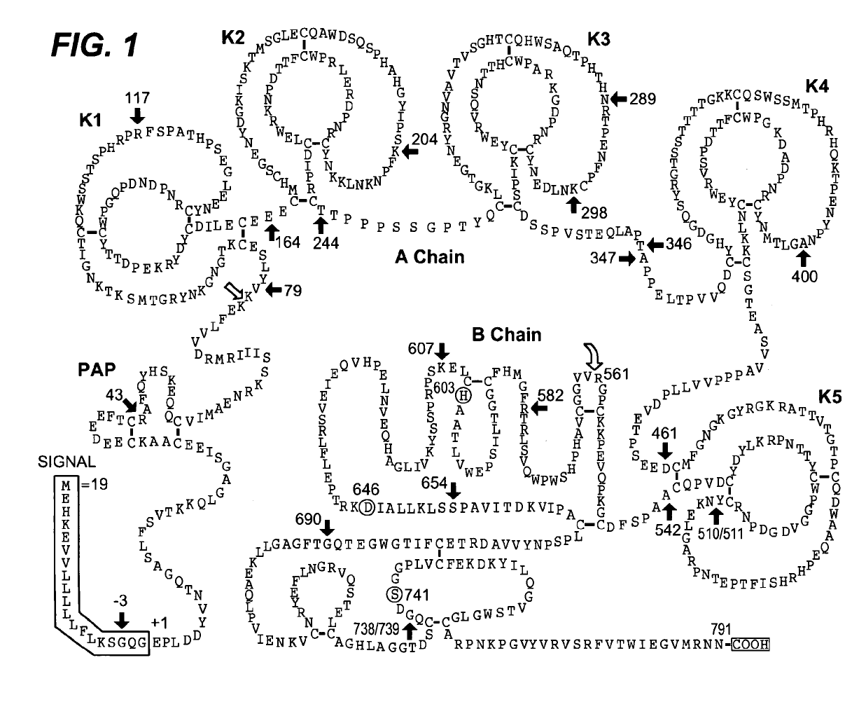

Full length naturally occurring human plasminogen with signal peptide is shown in SEQ ID NO:1 (numbering starts from the first amino acid of the pre-plasminogen), and FIG. 1 (numbering starts from the first amino acid of the mature plasminogen). Unless SEQ ID NO:1 is specified, amino acid positions are based on the numbering of the mature plasminogen. In some embodiments, the plasminogen polypeptide comprises the amino acid residues 561-810 (the catalytic domain) of SEQ ID NO:1. In some embodiments, one or more amino acid residues from 561-565 of SEQ ID NO:1 can be deleted. In some embodiments, the plasminogen polypeptide comprises the amino acid residues 458-810 of SEQ ID NO:1. In some embodiments, the plasminogen polypeptide comprises the amino acid residues 366-810 of SEQ ID NO:1. In some embodiments, the plasminogen polypeptide comprises the amino acid sequence of SEQ ID NO:3. In some embodiments, the plasminogen polypeptide comprises the amino acid residues 17-263 of SEQ ID NO:3. In some embodiments, the plasminogen polypeptide comprises the amino acid residues 17-267 of SEQ ID NO:3. In some embodiments, the plasminogen polypeptide comprises the amino acid sequence of SEQ ID NO:2. In some embodiments, the plasminogen polypeptide comprises the C-terminal amino acid sequence of SEQ ID NO:1 with kringles 4 and 5 and the catalytic domain (e.g., amino acid residues from about 366 to 810 of SEQ ID NO:1). In some embodiments, the plasminogen polypeptide comprises the C-terminal amino acid sequence of SEQ ID NO:1 with kringle 5 and the catalytic domain (e.g., amino acid residues from about 458 to 810 of SEQ ID NO:1).

Variants of plasminogen polypeptide of the present invention may include one or more amino acid substitutions, deletions or additions that do not significantly change the activity of the protein. Variants may be from natural mutations or human manipulation. Changes can be of a minor nature, such as conservative amino acid substitutions that do not significantly affect the folding or activity of the protein. To improve or alter the characteristics of the plasminogen polypeptides, protein engineering may be employed. Recombinant DNA technology known to those skilled in the art can be used to create novel mutant proteins or mutants including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Thus, plasminogen polypeptide also encompasses derivatives and analogs that have one or more amino acid residues deleted, added, or substituted to generate polypeptides that are better suited for expression, scale up, etc., in the host cells chosen. In some embodiments, amino acid sequences of the plasminogen variants are at least about any of 70%, 75%, 80%, 85%, 90%, 95%, 96%, 97%, 98%, or 99% identical to a naturally occurring plasminogen (such as from a human plasminogen).

Two polypeptide sequences are said to be “identical” if the sequence of amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e. the window size) and multiplying the results by 100 to yield the percentage of sequence identity.

Variants of plasminogen polypeptides also encompass fusion proteins comprising the plasminogen polypeptide. Biologically active plasminogen polypeptides can be fused with sequences, such as sequences that enhance immunological reactivity, facilitate the coupling of the polypeptide to a support or a carrier, or facilitate refolding and/or purification (e.g., sequences encoding epitopes such as Myc, HA derived from influenza virus hemagglutinin, His-6, FLAG, or the His-Tag shown in Table 3 of U.S. Pub. No. 2005/0227920). These sequences may be fused to plasminogen polypeptide at the N-terminal end or at the C-terminal end. In addition, the protein or polynucleotide can be fused to other or polypeptides which increase its function, or specify its localization in the cell, such as a secretion sequence. Methods for producing recombinant fusion proteins described above are known in the art. The recombinant fusion protein can be produced, refolded and isolated by methods well known in the art.

Variants of plasminogen polypeptides also include functional equivalent variants. Functional equivalent variants, are identified and characterized by any (one or more) of the following criteria: after being activated by a plasminogen activator, (a) ability to digest fibrin; b) ability to digest L-Pyroglutamyl-L-Phenylalanyl-L-Lysine-p-Nitroaniline hydrochloride or other serine protease substrates including natural and synthetic proteins or polypeptides. Biological activity of variants of plasminogen polypeptides may be tested using methods known in the art and methods described herein. In some embodiments, functional equivalent variants have at least about any of 50%, 60%, 70%, 75%, 80%, 85%, 90%, or 95% of activity as compared to full length native plasminogen with respect to one or more of the biological assays described above (or known in the art).

The invention also provides plasmin polypeptides generated by digestion of the peptide bond between arginine 561 and valine 562 (amino acid numbering is based on the numbering in FIG. 1) of native plasminogen polypeptides described herein.

### Methods of Producing Plasminogen and Plasmin Polypeptides

The methods of the invention are typically practiced utilizing inclusion bodies containing plasminogen polypeptide, such as plasminogen polypeptide produced in bacterial (e.g., E. coli) cells which have been engineered to produce the polypeptide, as the starting material, but any source of denatured plasminogen polypeptides may be used. The plasminogen may be from any species desired, and from any natural or non-natural plasminogen sequence, according to the practitioner's preference. The full length human plasminogen amino acid sequence including the signal sequence is shown in SEQ ID NO:1. Any polynucleotide sequences encoding the amino acid sequence may be used (such as changes which may improve expression in the host organism, i.e., “optimized” sequences).

Recombinant (e.g., bacterial, such as E. coli) host cells may be engineered to produce plasminogen polypeptide using any convenient technology. Most commonly, a DNA sequence encoding the desired plasminogen polypeptide is inserted into the appropriate site in a plasmid-based expression vector which provides appropriate transcriptional and translational control sequences, although expression vectors based on bacteriophage genomic DNA are also useful. It is generally preferred that the transcriptional control sequences are inducible by a change in the environment surrounding the host cells (such as addition of a substrate or pseudosubstrate to which the transcriptional control sequences are responsive), although constitutive transcriptional control sequences are also useful. As is standard in the art, it is also preferred that the expression vector include a positive selectable marker (e.g., the β-lactamase gene, which confers resistance to ampicillin) to allow for selection against bacterial host cells which do not contain the expression vector.

The bacterial host cells are typically cultured in a liquid growth medium for production of plasminogen polypeptide under conditions appropriate to the host cells and expression vector. Preferably, the host cells are cultured in a bacterial fermenter to maximize production, but any convenient method of culture is acceptable (e.g., shaken flask, especially for cultures of less than a liter in volume). As will be apparent to those of skill in the art, the exact growing conditions, timing and rate of media supplementation, and addition of inducing agent (where appropriate) will vary according to the identity of the host cells and the expression construct.

After the bacterial host cells are cultured to the desired density (and after any necessary induction of expression), the cells are collected. Collection is typically conveniently effected by centrifugation of the growth medium, although any other convenient technique may be used. The collected bacterial host cells may be washed at this stage to remove traces of the growth medium, most typically by resuspension in a simple buffer followed by centrifugation (or other convenient cell collection method). At this point, the collected bacterial host cells (the “cell paste”) may be immediately processed in accordance with the invention, or it may be frozen for processing at a later time.

The cells of the cell paste are lysed to release the polypeptide-containing inclusion bodies. Preferably, the cells are lysed under conditions in which the cellular debris is sufficiently disrupted that it fails to appear in the pellet under low speed centrifugation. Commonly, the cells are suspended in a buffer at about pH 5 to 9, preferably about 6 to 8, using an ionic strength of the order of about 0.01 M to 2 M preferably about 0.1-0.2 M (it is apparently undesirable to use essentially zero ionic strength). Any suitable salt, including NaCl can be used to maintain an appropriate ionic strength level. The cells, while suspended in the foregoing buffer, are then lysed by techniques commonly employed such as, for example, mechanical methods such as freeze/thaw cycling, the use of a Manton-Gaulin press, a French press, or a sonic oscillator, or by chemical or enzymatic methods such as treatment with lysozyme. It is generally desirable to perform cell lysis, and optionally bacterial cell collection, under conditions of reduced temperature (i.e., less than about 20° C.).

Inclusion bodies are collected from the lysed cell paste using any convenient technique (e.g., centrifugation), then washed. If desired, the collected inclusion bodies may be washed. Inclusion bodies are typically washed by resuspending the inclusion bodies in a wash buffer, typically the lysis buffer, preferably with a detergent added (e.g., 1% TRITON X-100®), then recollecting the inclusion bodies. The washed inclusion bodies are then dissolved in a solubilization buffer.

The solubilization buffer comprises a high concentration of a chaotroph, one or more reducing agents, and a buffer that buffers the solution to a pH of about 9.0 to about 11.0. The solubilization buffer may optionally contain additional agents, such as redox reagents, cation chelating agents and scavengers to neutralize protein-damaging free-radicals.

The instant invention utilizes urea as an exemplary chaotroph in the refolding buffer, although guanidine hydrochloride (guanidine HCl) may also be used. Useful concentrations of urea in the solubilization buffer include about 4 M to about 8 M, about 5 M to about 8 M, about 6 M to about 8 M, about 7 M to about 8 M. When the chaotroph is guanidine HCl, useful concentrations include about 1 M to about 8 M, or about 4 M to about 6 M, or about 6 M.

The pH of the refolding buffer is high, viz., in excess of pH 8.0, for example 10.0. For example, the pH may be of about 8.0 to about 11.0, about 9.0 to about 11.0, or about 9.0 to 10.0. As will be apparent to those of skill in the art, any pH buffering agent (or combination of agents) which effectively buffer at these pH ranges are useful, although pH buffers which can buffer in the range between pH 8.0 to pH 11.0 are particularly useful. Useful pH buffering agents include tris (tris(hydroxymethyl)aminomethane), bicine (N,N-Bis(2-hydroxyethyl)glycine), HEPBS (2-Hydroxy-1,1-bis[hydroxymethyl]ethyl)amino]-1-propanesulfonic acid), TAPS ([(2-Hydroxy-1,1-bis[hydroxymethyl]ethyl)amino]-1-propanesulfonic acid), AMPD (2-Amino-2-methyl-1,3-propanediol).N-(2-Hydroxyethyl)piperazine-N′-(4-butanesulfonic acid)), and the like. The pH buffering agent is added to a concentration that provides effective pH buffering, such as from about 10 mM to about 400 mM, about 75 mM to about 300 mM, about 200 mM, or about 20 mM.

Reducing agents are included in the solubilization buffer to reduce disulfide bonds and maintain cysteine residues in their reduced form. Useful reducing agents include β-mercaptoethanol, dithiothreitol, and the like. More than one reducing agents, such as both β-mercaptoethanol and dithiothreitol, may be used. Additionally, the refolding buffer may contain disulfide reshuffling or “redox” reagents (e.g., a combination of oxidized and reduced glutathione). When the redox reagents are oxidized and reduced glutathione (GSSG and GSH, respectively), useful concentrations include about 0.1 mM to about 10 mM and useful ratios include about 10:1, about 5:1, and about 1:1 (GSH:GSSG).

The solubilization buffer may contain additional components. For example, the solubilization buffer may contain a cation chelator such as a divalent cation chelator like ethylenediaminetetraacetic acid (EDTA) or ethylene glycol-bis(2-aminoethylether)-N,N,N′,N′-tetraacetic acid (EGTA). EDTA or EGTA is added to the solubilization buffer at a concentration of about 0.5 to about 5 mM, and commonly at about 1 mM. Additionally, a free-radical scavenger may be added to reduce or eliminate free-radical-mediated protein damage, particularly if urea is used as the chaotroph and it is expected that a urea-containing protein solution will be stored for any significant period of time. Suitable free-radical scavengers include glycine (e.g., at about 0.5 to about 2 mM, or about 1 mM) and other amino acids and amines.

An exemplary solubilization buffer comprises about the following concentrations of the following components: 8 M urea, 100 mM Tris, 1 mM glycine, 1 mM EDTA, 10 mM beta-mercaptoethanol, 10 mM dithiothreitol (DTT), 1 mM reduced glutathion (GSH), 0.1 mM oxidized glutathion (GSSG), pH 10.0.

The inclusion body/solubilization buffer mixture is incubated to allow full solubilization. The incubation period is generally from about 6 hours to about 24 hours, and more commonly about eight to about 8 hours or about 16 hours, or about 12 hours. Stirring may be applied, for example at a speed of 500 rpm. The inclusion body/solubilization buffer mixture incubation may be carried out at room temperature or at reduced temperature. For example, temperature may be between about 4° C. to about 10° C.

After the incubation is complete, the inclusion body/solubilization buffer mixture is clarified to remove undissolved inclusion bodies. Clarification of the mixture may be accomplished by any convenient means, such as filtration (e.g., by use of depth filtration media) or by centrifugation, or both. Clarification should be carried out at reduced temperature, such as at about 4° to about 10° C.

The clarified mixture is then diluted using the same solubilization buffer to achieve the appropriate protein concentration for refolding. Protein concentration may be determined using any convenient technique, such as Bradford assay, light absorption at 280 nm (A₂₈₀), and the like. A solution having from about 0.5 mg/ml to about 10 mg/ml (e.g., about 2 mg/ml) is appropriate for use in the instant methods. If desired, this mixture may be held, refrigerated (e.g. at 4° C.), for later processing, although the mixture is not normally held for more than about four weeks.

The concentration-adjusted inclusion body solution is first rapidly diluted about 20 fold with a refolding buffer. The dilution is performed by adding inclusion body solution into the refolding buffer. The inclusion body solution may be diluted about 5 to about 100 fold, about 10 to about 50 fold, about 10 to about 25 fold, about 15 to about 25 fold with the refolding buffer. The inclusion body solution is diluted to reduce urea and protein concentration. The final protein concentration after dilution may be about 0.01 mg/ml to about 1 mg/ml, about 0.1 mg/ml to about 0.5 mg/ml. The concentration of urea or guanidine HCl in the diluted inclusion body solution may be about 1 M to about 3 M for urea; and about 0.5 M to about 2 M for guandine HCl. This concentration of urea or guanidine HCl may be achieved by just diluting the inclusion body/solubilization buffer, or added into the refolding buffer.

The refolding buffer contains a pH buffer and arginine. The refolding buffer may also contain a low concentration of chaotroph, a disulfide reshuffling reagent, and a divalent cation chelator. These reagents may be added to the refolding buffer. The refolding buffer may include additional agents, such as free-radical scavengers. “Rapid” dilution, within the context of the invention means over a period of less than about 25 minutes, and the dilution process is generally carried out during periods of about two minutes to about 25 minutes, or about five to about 20 minutes. The diluted solubilized plasminogen polypeptide solution is typically held for one to two hours following the completion of the rapid dilution process.

The refolding buffer may contain about 0.05 M to about 0.5 M arginine. The refolding buffer may also contain glycerol (e.g., about 5 to about 20%) or sucrose (about 5 to about 30%).

The pH of the refolding buffer may be the same as or different from the solubilization buffer. The pH of the refolding buffer may be from about 8.0 to about 10.0, from about 8.5 to about 9.5, or from about 9.0 to about 9.5. In some embodiments, the refolding buffer has a pH of about 9.0 (e.g., for refolding miniplasminogen). In some embodiments, the refolding buffer has a pH of about 9.5 (e.g., for refolding microplasminogen). The pH buffering agent in the refolding buffer may be any buffering agent or combination of buffering agents that are effective pH buffers at pH levels of about 8 to about 9 or about 10 or about 10.5. Useful pH buffering agents include tris (tris(hydroxymethyl)aminomethane), bicine (N,N-Bis(2-hydroxyethyl)glycine), HEPBS (2-Hydroxy-1,1-bis[hydroxymethyl]ethyl)amino]-1-propanesulfonic acid), TAPS ([(2-Hydroxy-1,1-bis[hydroxymethyl]ethyl)amino]-1-propanesulfonic acid), and AMPD (2-Amino-2-methyl-1,3-propanediol).N-(2-Hydroxyethyl)piperazine-N-(4-butanesulfonic acid)). The pH buffering agent is added to a concentration that provides effective pH buffering, such as from about 10 to about 150 mM, about 50 to about 150 mM, about 75 mM to about 125 mM, or about 100 mM.

The redox reagents included in the refolding buffer must be effective in ‘shuffling’ cysteine sulfhydryl groups between their oxidized and reduced states. The redox environment of the refolding reaction may be adjusted by manipulating the concentration of the redox reagents. When the redox reagents are oxidized and reduced glutathione (GSSG and GSH, respectively), the inventor has found that useful concentrations include about 0.005 mM to about 0.05 mM, about 0.1 mM to about 11 mM and useful ratios include about 10:1, about 5:1, and about 1:1 (GSH:GSSG).

The divalent cation chelator may be any molecule that effectively chelates Ca⁺⁺ and other divalent cations. Exemplary cation chelators for use in the refolding buffer include ethylenediaminetetraacetic acid (EDTA) or ethylene glycol-bis(2-aminoethylether)-N,N,N′,N′-tetraacetic acid (EGTA). When EDTA or EDTA is the divalent cation chelator, it is added to the refolding buffer at a concentration of about 0.5 to about 5 mM, and commonly at about 1 mM.

Additional components useful in the refolding buffer include free-radical scavengers. A free-radical scavenger may be added to reduce or eliminate free-radical-mediated protein damage, particularly if urea is used as the chaotroph and it is expected that a urea-containing protein solution will be stored for any significant period of time. Suitable free-radical scavengers include glycine (e.g., at about 0.5 to about 2 mM, or about 1 mM).

The refolding buffer may further comprise a reversible protease inhibitor, such as PMSF.

An exemplary refolding buffer comprises Tris and arginine at pH about 9.0 or about 9.5. In some embodiments, the refolding buffer comprises about 20 mM Tris and about 0.2 M arginine at pH about 9.5. In some embodiments, the refolding buffer comprises about 20 mM Tris, about 0.2 M arginine, about 0.02 mM PMSF, at pH about 9.0.

The refolding reaction is incubated for a period of about 1 to 2 hours to about 18 to 24 hours. The refolding reaction may be carried out at room temperature (e.g., about 18-20° C.) or at slightly reduced temperature (e.g., about 14-16° C.), depending on the preferences of the practitioner and the availability facilities.

Following the refolding, properly refolded plasminogen polypeptide may be concentrated, buffer exchanged, and further purified. Concentration/buffer exchange of the refolded protein may be accomplished using any convenient technique, such as ultrafiltration, diafiltration, chromatography (e.g., ion-exchange, hydrophobic interaction, or affinity chromatography) and the like. Where practical, it is preferred that concentration be carried out at reduced temperature (e.g., about 4-10° C.).

While any convenient protein purification protocol may be used. In some embodiments, two types of chromatography may be used for purification. For example, size exclusion chromatography (SEC) and affinity chromatography may be used.

Size exclusion chromatography (SEC) may be performed using any convenient chromatography medium which separates properly folded plasminogen polypeptide from unfolded and multimeric form. The inventor has found that media having the ability to size fractionate proteins of about 10⁴ to about 6×10⁵ daltons (globular proteins) are useful for this step. Exemplary SEC media include Sephacryl® 300, Superdex™ 200, and Superdex™ 75. This step may also be used to perform buffer exchange, if so desired. The exact conditions for SEC will depend on the exact chromatography media selected, whether buffer exchange is to be accomplished, the requirements of any later purification steps, and other factors known to those of skill in the art.

The properly folded plasminogen polypeptide may be further purified utilizing affinity chromatography, for example, benzamidine affinity column (e.g., obtained from GE Amersham Biosciences).

Cation exchange chromatography (e.g., sulfopropyl ion exchange chromatography) may be used for purification. Cation exchange chromatography may be washed with a buffer containing 20 mM citrate, 10% sucrose, pH 3.1, with 0-1 M NaCl gradient.

Refolded plasminogen polypeptide can be treated with plasminogen activator, such as urokinase, tissue plasminogen activator (tPA), streptokinase, staphylokinase, to generate biologically active plasmin, which may be further purified using any of the methods described above for plasminogen or methods known in the art.

Biological activity of plasmin polypeptide produced from the properly folded recombinant plasminogen polypeptide produced in accordance with the invention may be measured using any acceptable assay method known in the art. An exemplary method of measuring plasmin activity is described herein in Examples, which measures amidolysis of plasmin chromogenic substrate S-2403 (L-Pyroglutamyl-L-Phenylalanyl-L-Lysine-p-Nitroaniline hydrochloride).

The invention also provides a composition (e.g., an aqueous composition) comprising a plasminogen or plasmin polypeptide produced by the method described herein. In some embodiments, the composition may further comprise a pharmaceutically acceptable carrier, excipient, or stabilizer (Remington: The Science and Practice of Pharmacy 20th Ed. (2000) Lippincott Williams and Wilkins, Ed. K. E. Hoover.), in the form of lyophilized formulations or aqueous solutions. Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are known in the art.

Purified plasmin polypeptide may be stored in low pH buffer with sucrose. For example, citric acid at pH of less than about 5.0 or less than about 4.0, or about 3.1 may be used. An exemplary buffer comprises about 20 mM citric acid, about 7.5% sucrose, about 0.15 M NaCl, at pH about 3.1.

As is well understood in the art, all concentrations and pH values need not be exact and reference to a given value reflects standard usage in the art, does not mean that the value cannot vary.

The following examples provide a detailed description of the production of properly folded recombinant plasminogen and plasmin polypeptides in accordance with the methods of the invention and the characterization thereof. These examples are not intended to limit the invention in any way.

### Examples

### Materials and Methods

Construction of μPlg and miniPlg expression vector: Synthetic cDNAs encoding μPlg and miniPlg optimized for expression in E. coli were synthesized by CODA Genomics (Laguna Hills, Calif.). The protein sequences used to generate the cDNAs were obtained from the protein sequence for full length human plasminogen (Accession # P00747) from the ExPASY server (www.expasy.org) of the Swiss Institute of Bioinformatics. The optimized cDNA encoding μPlg was inserted into the unique BamH1/Xho1 sites of a custom pET11a expression vector with a modified expanded multiple cloning site, creating the plasmid pET11a-μPlg. The optimized cDNA encoding miniPlg was inserted into the same vector but at the unique NdeI/BamH1 sites to create expression vector pET11a-miniPlg. The final clones were DNA sequence verified (MWG, High Point, N.C.).

Expression of μPlg and miniPlg in E. coli: Expression of μPlg and miniPlg was carried out using the auto-induction method developed by Studier at Brookhaven National Laboratory (9). E. coli BL21 (DE3) cells were transformed with either pET11a-μPlg or pET11a-miniPlg and plated onto PA-0.5G/Ampicillin plates and incubated for 16 hours at 37° C. One colony was used to inoculate 50 ml PA-0.5G liquid media and incubated in a shaking flask at 37° C. for 16 hours to an OD₆₀₀ of 5-6 and immediately stored at 4° C. to create a working stock. The working stock (0.5 ml) is used to inoculate 500 ml ZYP-5052 liquid media and incubated 16 hours at 37° C., 300 RPM to an OD₆₀₀ of 8-10 in a 2.8 L Fernbach flask. For larger scale production, 6-12 L was produced in multiple 2.8 L Fernbach shaker flasks.

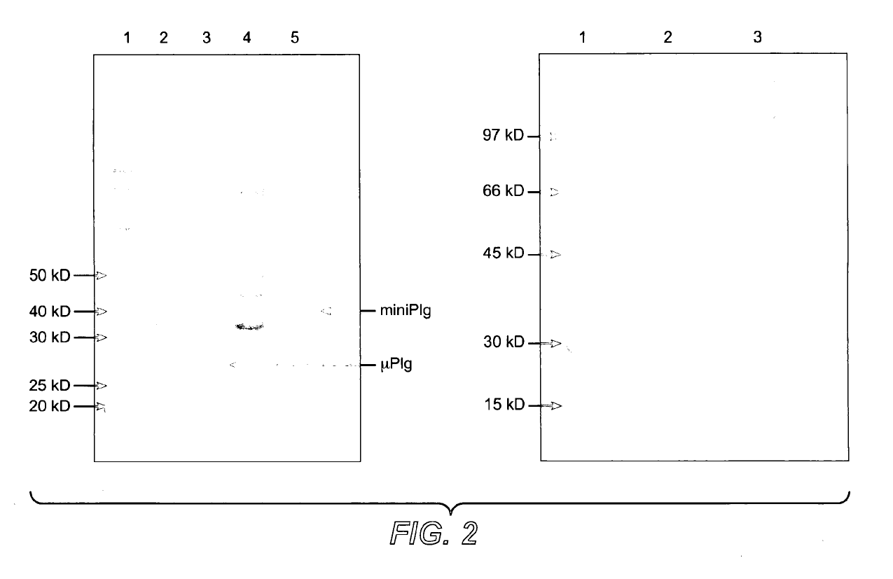

For the data in FIG. 2 only, 3 ml cultures of pET11a-μPlg/BL21(DE3) and pET11a-miniPlg/BL21(DE3) were grown in LB amp broth to OD600=0.6. IPTG was then added at a final concentration of 1 mM and the culture was induced for 3 hrs at 37° C. The samples were then spun down and the pellets were suspended in SDS running buffer with added 100 mM β-ME and loaded onto SDS PAGE for analysis. Controls without IPTG were run in parallel.

Isolation and Solubilization of Inclusion Bodies: After induction, the cells were harvested and centrifuged at 7,000 RPM, 4° C. for 10 minutes. The pellet was then resuspended in 50 ml of 50 mM Tris, 25% sucrose, 1 mM EDTA, 10 mM DTT, pH 8.0. Lysozyme (100 mg) was added and the solution was stirred for 30 minutes at room temperature. A buffer (125 ml) containing 20 mM Tris, pH 7.5, 1% NaDeoxycholate, 1% Triton X-100, 10 mM NaCl, 10 mM DTT was added and the solution was stirred for an additional 30 minutes and then frozen at −85° C. for 20 hours. The solution was then thawed in a 37° C. water bath for 3 hours and homogenized using a Branson ultrasonic horn at 70% amplitude, 30 second pulse, 10 second pause for 6 cycles. The solution was brought to 1 L with 50 mM Tris, 100 mM NaCl, 0.5% Triton X-100, 1 mM EDTA, 1 mM DTT, pH 8.0 (Triton Solution) and stirred at 800 RPM, 4° C. for 1 hour. The inclusion bodies were then centrifuged at 7,000 RPM, 4° C. for 1 hour, and the resulting pellet was resuspended with 1 L of Triton Solution and stirred again at 800 RPM, 4° C. for 1 hour. The cycle of centrifugation and resuspension in Triton Solution was repeated 3 more times followed by 4 washes with 50 mM Tris, 1 mM EDTA, 1 mM DTT (Tris Solution). After the last wash with Tris Solution, the pellet was solubilized in 10 ml 8M Urea, 100 mM Tris, 1 mM Glycine, 100 mM Beta-ME, pH 10.5 and stirred for 16 hours at 300 RPM, 4° C. The solubilized inclusion bodies were then ultracentrifuged at 30,000 RPM, 4° C. for 30 minutes and characterized by A₂₈₀ and non-reducing SDS-PAGE.

Refolding of denatured μPlg and miniPlg: The purified inclusion body preps were diluted in a 8 M urea buffer (8 M urea, 0.1 M Tris, 1 mM glycine, 1 mM EDTA, 10 mM β-ME, 10 mM dithiothreitol (DTT), 1 mM reduced glutathione (GSH), 0.1 mM oxidized glutathione (GSSG), pH 10 with a final concentration of 2 mg/ml as an initial step just before rapid dilution refolding was initiated. The inclusion body was incubated from 10 min to about 2 hours. Subsequently, for μPlg, this solution was rapidly diluted into 20 volumes 20 mM Tris, 0.2 M arginine, pH 9.5. For miniPlg, this solution was rapidly diluted into 20 volumes 20 mM Tris, 0.2 M arginine, 0.02 mM PMSF, pH 9.0. The final volume in the experiments ranged from 4 L to 16 L. The solutions were allowed to refold over night at 18° C. (approximately 16 hrs) and then were transferred to a 4° C. cold room for an additional 48 hrs.

Purification Procedure: For μPlg, purification was performed by first concentrating the refolding buffer to 0.5 L using tangential flow filtration through 3 Kvick Lab 10K membranes (Amersham, Piscataway, N.J.). The retentate was dialyzed against 20 mM MES, 10% Sucrose, pH 6.5 overnight at 4° C. The solution was centrifuged at 20K RPM, 4° C. for 30 minutes, and the supernatant was loaded onto a 150 ml SP Sepharose Fast Flow column (Amersham, Piscataway, N.J.) equilibrated with three column volumes of buffer A: 20 mM MES, 10% Sucrose, pH 6.5. Elution was performed using a liner gradient from 0% to 100% of buffer B: 20 mM Tris, 10% Sucrose, 1M NaCl, pH 6.5 at 5 ml/min over 6 column volumes at room temperature with 10 ml fractions being collected. Peak fractions were combined and were then dialyzed against 20 mM Citric Acid, 10% Sucrose, pH 3.1 overnight at 4° C. The solution was concentrated to 10 ml using high pressure nitrogen filtration through a Millipore Amicon 10K membrane (Millipore, Bedford, Mass.). The retentate was centrifuged at 30K RPM, 4° C. for 30 minutes and loaded onto a Superdex 75 column (XK50x850-mm, Amersham, Piscataway, N.J.) equilibrated with 2 column volumes of 20 mM Citric Acid, 7.5% Sucrose, 0.15M NaCl, pH 3.1. The sample was eluted by running 1 column volume of 20 mM Citric Acid, 10% Sucrose, 0.15M NaCl, pH 3.1 buffer at 4° C. with 10 ml fractions being collected. Peak fractions were identified by A₂₈₀ and non-reducing SDS PAGE and were then pooled and stored at −85° C. The yield of isolated fractions was computed by molar extinction in 6M guanidine, 38 mM NaPi, pH 6.5, where 1 mg/mL=1.40 A₂₈₀.

For miniPlg, purification was performed by first concentrating the refolding buffer to 0.5 L using tangential flow filtration using Kvick Lab 10K membranes. The material was further concentrated to 20 ml using the Millipore Amicon N₂ pressure membrane concentrator, ultracentrifuged at 30K RPM, 4° C. for thirty minutes and then loaded onto a Superdex 75 size exclusion column (XK50x850-mm, Amersham, Piscataway, N.J.) equilibrated with 20 mM Tris, 0.2 M arginine, 0.15 M NaCl, 0.02 mM PMSF, pH 9.0 at 4° C. Fractions (10 ml) were collected and analyzed by A₂₈₀ and SDS-PAGE under non-reducing conditions. If necessary for “polishing”, peak monomeric fractions (Mr˜38 KDa) were pooled and reconcentrated using the N₂ pressure membrane concentrator and loaded onto a Superdex 200 column (XK26x850-mm, Amersham, Piscataway, N.J.) equilibrated with 20 mM Citric Acid, 10% Sucrose, 0.15M NaCl, pH 3.1 at 4° C. Peak fractions were identified at A₂₈₀ and by non-reducing SDS-PAGE and were then pooled and stored at −85° C. The yield of isolated fractions was computed by molar extinction in 6M guanidine, 38 mM NaPi, pH 6.5, where 1 mg/mL=1.54 A₂₈₀.

Kinetic Characterization of Refolded Molecules: Refolded μPlm and miniPlm were compared kinetically with commercially available plasmin (Sigma, St Louis, Mo.) at 21° C. using the commercially available plasmin chromogenic substrate 5-2403 (L-Pyroglutamyl-L-Phenylalanyl-L-Lysine-p-Nitroaniline hydrochloride) (Chromogenix, Goteberg, Sweden). As an initial step, appropriate quantities of μPlg and miniPlg were diluted to 200 μg/mL in 100 mM Tris, 150 mM NaCl, 0.01% Tween 20, pH 7.6. Urokinase was added at a molar stoichiometric ratio of 1:20 (urokinase:Plg) and incubated for 15 minutes at 37° C. to convert the zymogens to μPlm or miniPlm. 10 μL aliquots of μPlm, miniPlm, or plasmin were then rapidly mixed with 100 μL aliquots of S-2403 in 50 mM Tris, 150 mM NaCl, 0.01% Tween20, pH 7.6 at a range of concentrations of S-2403 (300-3000 μM) in quadruplicate using a titertek micropipetter in a 96-well microplate. The experiment was performed in the absence of fibrin or fibrinogen. The urokinase used to activate the plasmins does not catalyze amidolysis of S-2403. The A₄₀₅ was measured at 15 second intervals for 5-15 minutes using a Spectramax 384 plate reader (Molecular Devices, Sunnyvale, Calif.). The raw data was converted to μM amounts and analyzed using a Hanes plot.

Amino Acid Sequencing: N-terminal amino acid sequencing was performed on individual protein bands excised from PVDF membrane protein blots (Millipore, Bedford, Mass.) at Molecular Biology Resource Facility, University of Oklahoma Health Sciences Center, Oklahoma City, Okla.

### Results:

High level expression of μPlg and miniPlg as inclusion bodies: FIG. 2A depicts specific expression of μPlg and miniPlg expression in the presence and absence of IPTG. FIG. 2B depicts purified inclusion bodies for μPlg and miniPlg. The proteins in the inclusion bodies are estimated to be at least 90% pure before refolding is initiated. Although the data shown in FIG. 2A involved growth in LB broth and induction with IPTG, auto-induction system of Studier (9) was routinely used for large scale preparation of inclusion bodies, since a 6-10 fold higher yields of inclusion bodies were obtained. When the inclusion bodies were purified, as described in Materials and Methods, a final yield of 500-600 mg of inclusion bodies was obtained per liter of ZYP-5052 expression medium for both μPlg and miniPlg.

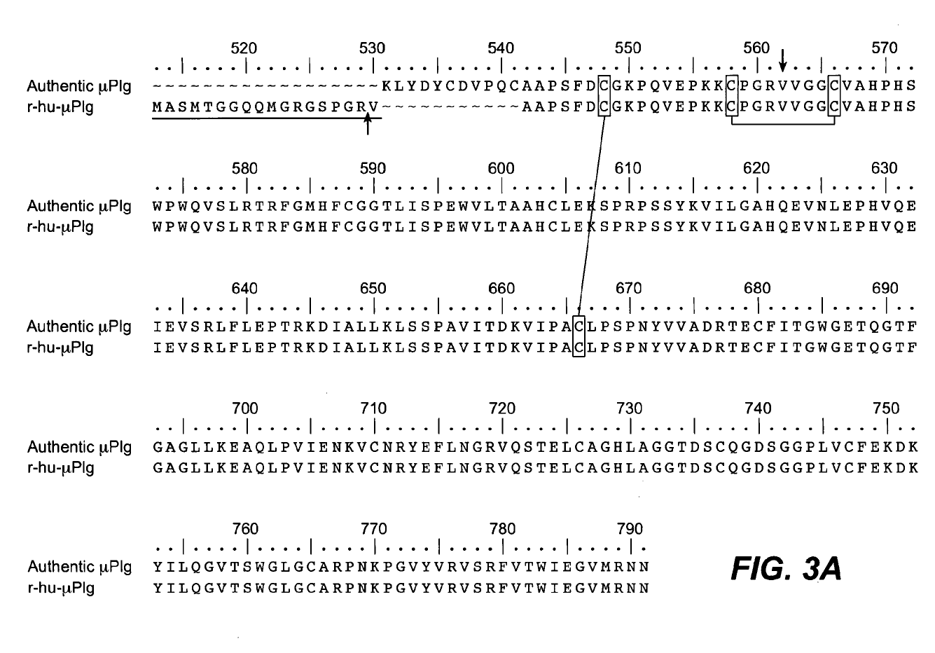

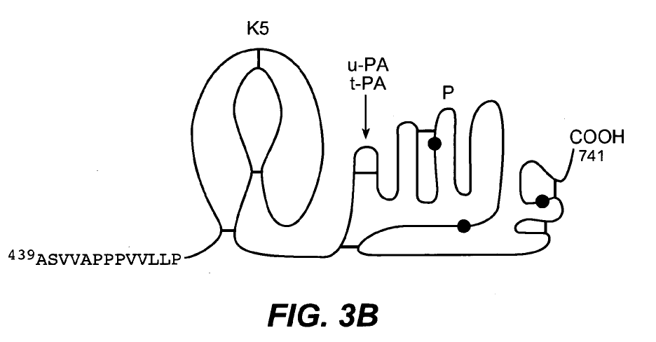

Primary Protein Structure of μPlg and miniPlg Expression Constructs: Microplasmin produced from plasmin by alkaline treatment (3, 4) results in a 31 amino acid A chain and a 230 amino acid B chain (FIG. 3A, authentic μPlg). The numbering system used in the figure uses the nomenclature for full length plasminogen. Cysteines 548 and 558 each form essential disulfide bridges with cysteines in the B chain but amino acids 531-545 can be deleted without effect on function (5). A recombinant form of single chain μPlg, designated as r-hu-μPlg in FIG. 3A, was constructed. This protein was expressed as pET11a-μPlg as described in Materials and Methods. The r-hu-μPlg is a fusion protein with an extra N-terminal 18 amino acid (FIG. 3A, underlined) for facilitating expression of inclusion bodies in E. coli. Treatment of r-hu-μlg with plasminogen activator such as urokinase results in cleavage not only at the authentic activation site, the peptide bond between arginine 561-valine 562 (R^{561∇}V⁵⁶²), but also at an artificial site at the junction between the artificial amino terminus and the authentic amino acids of chain A. Cleavage at this site, indicated as a vertical pointing arrow in FIG. 3A, trims away all but a valine residue of the artificial amino terminus (GR^{∇}VA⁵⁴²) when the molecule is activated to μPlm. In a previous publication, Reich and coworkers (5) established that removal of the amino acids prior to serine 545 in Chain A was not detrimental to the catalytic properties of μPlm.

A schematic diagram of miniPlg is presented in FIG. 3B. The original miniPlg was generated by neutrophil elastase digestion of plasmin at the peptide bond between valine 441 and valine 442 (1, 2). The recombinant miniPlg in this example started from alanine 439, as indicated in FIG. 3B. N-terminal amino acid sequencing of the purified miniPlg indicates that the starting amino acid methionine is removed during expression.

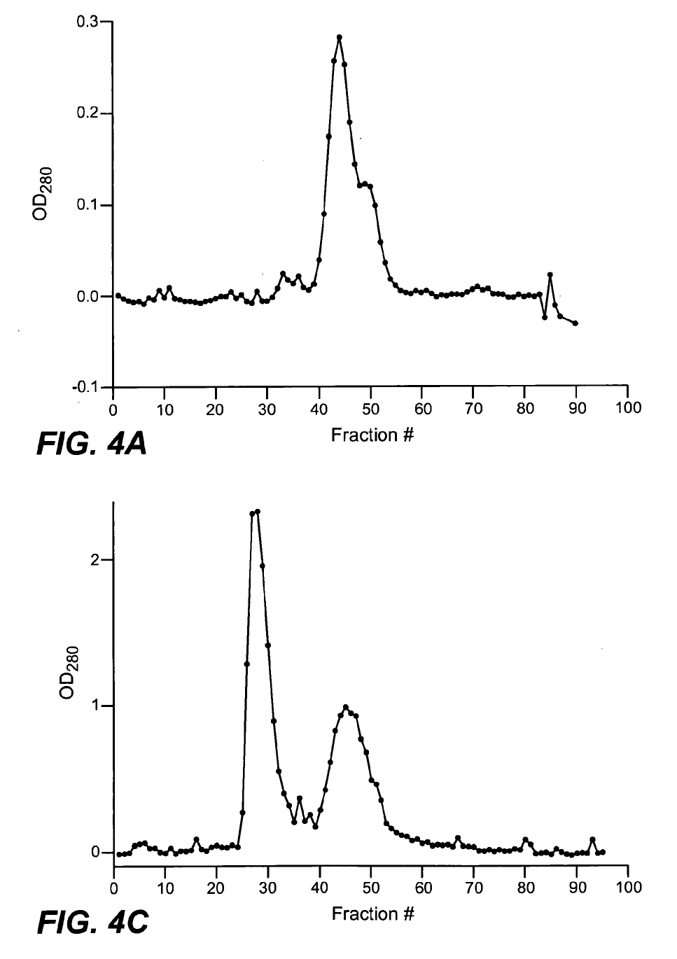

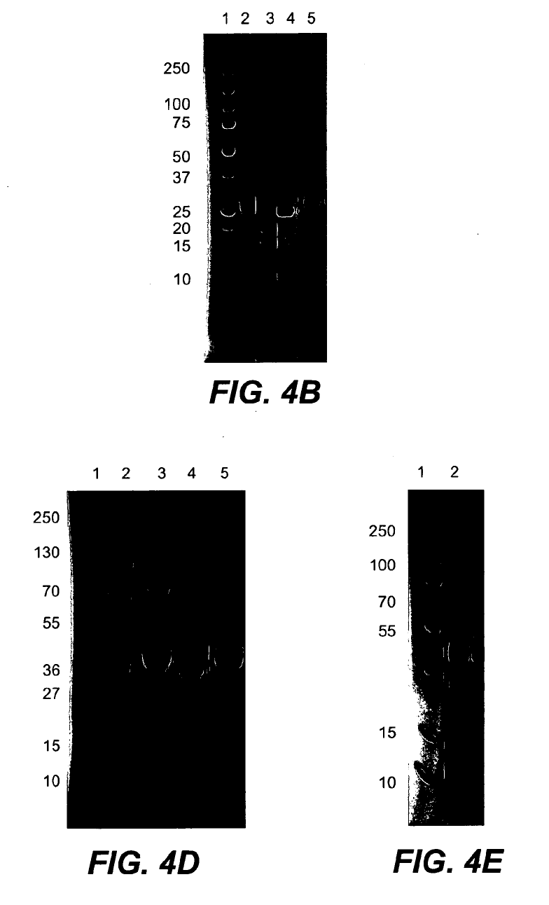

Refolding and Purification of μPlg and miniPlg: Microplasminogen has six disulfide bridges and miniPlg has nine disulfide bridges. Therefore, it is not surprising that there would be a significant amount of incorrectly formed disulfides and multimeric forms of the protein generated during the refolding process. Before arriving at final refolding conditions, described in the Materials and Methods section and in Table 1, a small scale “screening refolding” process consisting of a matrix was conducted, and more than 40 refolding conditions were examined. Criterion for selection in the initial screening included lack of visible protein precipitate during refolding or in subsequent concentration steps for SEC, and elution of protein peaks from the SEC with a mobility identical to refolded monomeric forms of the protein. SEC is used as an important tool in post-refolding purification because multimeric, unfolded, soluble forms of the protein can be easily separated from refolded monomer. Ultimately, the refolding conditions summarized in Table 1 were selected because they generated the highest percent of monomeric form of μPlg or miniPlg in the SEC chromatographic step. They also presented a favorable activity profile when the proteins were activated to either μPlm or miniPlm and examined in a functional assay (discussed below). Representative chromatograms of isolated μPlg and miniPlg are presented in FIGS. 4A and 4C, respectively. Overall, approximately 10% of total protein refolded from inclusion bodies, estimated to be >90% pure at the outset, was isolated in the final monomeric refolded form at >98% purity (Table 1, FIGS. 4B, 4D). Because of the very high yield of inclusion bodies using the T7 promoter and the auto-induction expression system of Studier (9), the estimate for the production yield is at least 50-60 mg of purified, fully function, monomeric μPlm or miniPlm from each liter of expression culture using Fernbach shaker flasks.

TABLE 1
Summary of Refolding Protocol for μPlasminogen and Miniplasminogen

| Protein | μPlasminogen | miniPlasminogen |
|---|---|---|
| Refolding Buffer | 20 mM Tris, 0.2 M Arginine | 20 mM Tris, 0.2 M Arginine 0.02 mM PMSF |
| | 0.4 M urea, 0.05 mM GSH, 0.005 mM GSSG, | 0.4 M urea, 0.05 mM GSH, 0.005 mM GSSG, |
| | 0.5 mM β-ME, 0.05 mM EDTA pH 9.5 | 0.5 mM β-ME, 0.05 mM EDTA pH 9.0 |
| Refolding Concentration | 0.1 mg/mL | 0.1 mg/mL |
| Refolding Time | Overnight at 18° C., | Overnight at 18° C., |
| | then 48 hrs at 4° C. | then 48 hrs at 4° C. |
| Total Protein Monomer | 20-25% after SP IEX | 15-20% after Superdex 75 SEC |
| Yield After 1^{st} Step of Purification | chromatography | |
| Total Protein Monomer Yield | 10% after Superdex 75 SEC | 10% after Superdex 200 SEC |
| After 2^{nd} Step of Purification | | |

Autoactivation: After isolating monomeric correctly refolded μPlg and miniPlg, it was discovered that both of these molecules have a tendency to auto-activate to μPlm and miniPlm respectively if the isolated fractions were left at neutral or alkaline pH at 4° C. over several days. Unlike the zymogen form, the auto-activated material displayed full enzymatic activity. The activation occurs without the addition of plasminogen activator, urokinase. The actual auto-activation could be detected clearly after SEC by SDS-PAGE if samples were reduced with β-ME. In the case of μPlg was clearly demonstrated when material isolated after a first column purification step (IEX, sulfopropyl ion exchange column) was left at neutral pH for several days before running a second purification step (SEC column). Analysis of the SEC column (FIG. 4A) fractions by SDS-PAGE (FIG. 4B) indicated separation into 2 peaks—one of auto-activated material (FIG. 4B, lanes 2, 3) and one of unactivated zymogen (FIG. 4B, lanes 4, 5). The auto-activated material was not visible without reduction because of the interconnection by disulfide bonds (FIG. 4B, lanes 2, 4). Interestingly, unactivated reduced μPlg displayed a reduced mobility on the gel relative to its non-reduced state (FIG. 4B lanes 5 vs. lane 4), probably because the polypeptide backbone of the protein linearized after reduction. N-terminal amino acid sequencing of PVDF protein blots from the SDS-PAGE bands confirmed that the auto-activation occurred at R^{561∇}V⁵⁶², which is the authentic activation site for plasminogen activator. The auto-activation could be prevented completely if the pH was lowered immediately either by dialysis or by adding solid sodium citrate and sucrose (final buffer concentration: 20 mM citrate, pH 3.1, 10% sucrose). This observation parallels that made by Jensen (10) three decades ago for stabilizing full length plasmin. By including the rapid addition of sucrose and citrate into the isolated fractions and lower the pH to 3.1 in our protocol as described in Material and Methods, the auto-activation of μPlg presented in FIG. 4B can be prevented.

Using similar method for μPlm purification, the citrate/sucrose buffer was also incorporated into the separation procedure when purifying the refolded miniPlm. As is demonstrated in FIG. 4C, during the first step of chromatography, SEC, two peaks were isolated. The first peak contained soluble, misfolded, high molecular weight multimers of miniPlg (FIG. 4D, lanes 2, 3) while the second peak consisted primarily of the monomeric form (FIG. 4C, lanes 4, 5). Solid citrate and sucrose were added to the column fractions immediately after isolation to prevent auto-activation. The purified material could be stored at 4° C. in citrate buffer at pH 3.1 for more than 30 days without evidence of auto-activation, and could also be frozen without loss of activity. However, if the material was re-elevated to neutral pH, auto-activation would occur (FIG. 4E, lane 2).

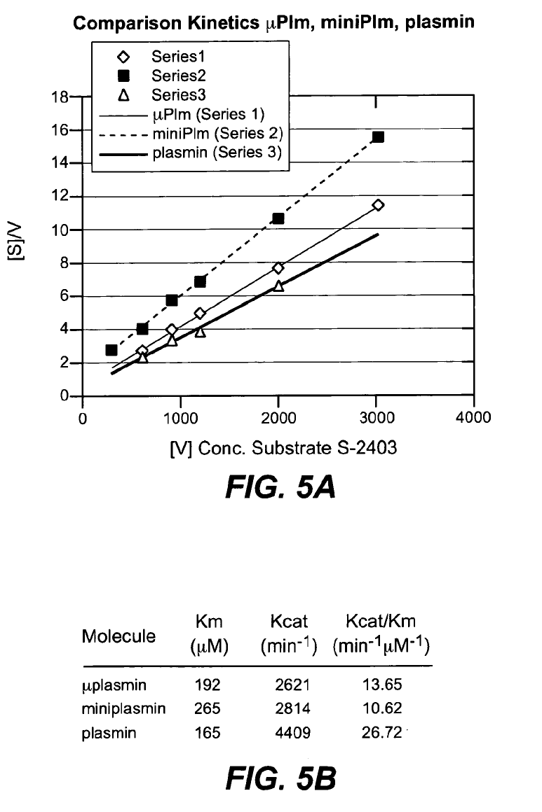

Kinetic Characterization of μPlm and miniPlm: Refolded μPlm and miniPlm were compared kinetically with commercially available plasmin at 21° C. using plasmin chromogenic substrate S-2403. As described in detail in Materials and Methods, a fixed concentration of μPlm, miniPlm, or plasmin was mixed with a range of concentrations of S-2403 (300-3000W). As summarized in FIG. 5, the kinetic parameters for the three molecules were relatively similar. Plasmin had the lowest Km (165 μM) and the highest Kcat (4409 min⁻¹); its catalytic efficiency was about 2× higher than μPlm and about 2.6× higher than miniPlm toward the synthetic substrate. These initial data establish that the active site conformation of the serine protease domain of the three molecules is very similar, validated the refolding methodology. However, it is well known that fibrin interacts with the kringle domains of plasmin and miniPlm, significantly increasing the Kcat (11-13) relative to μPlm. No comparison has been made between these 3 enzymes using fibrin as a competing substrate.

In summary, this example demonstrated that large quantities of μPlg and miniPlg can be produced as inclusion bodies in E. coli, and refolded to functionality. The auto-induction protein expression system developed by Studier (9) in conjunction with expression plasmids containing an T7 promoter controlled by a lac operon has been used to generate high-level expression of inclusion bodies in a shaker flask expression system. The quantity of μPlg or miniPlg produced as inclusion bodies exceeds that using a LB broth/IPTG inducible T7 expression system by 6-10 folds in these specific cases. The methods described in this example produced 500-600 mg/L of purified inclusion bodies routinely for μPlg and miniPlg, and the final recovery of functionally active monomeric protein after refolding and purification—about 10%—represent at least 50 mg of recombinant products.

### Amino Acid Sequences for Human Plasminogen Polypeptides:

| Human full length pre-plasminogen sequence (SEQ ID NO: 1): | |
|---|---|
| 1 | mehkevvlll llflksgqge plddyvntqg aslfsvtkkq lgagsieeca akceedeeft |
| 61 | crafqyhske qqcvimaenr kssiiirmrd vvlfekkvyl secktgngkn yrgtmsktkn |
| 121 | gitcqkwsst sphrprfspa thpsegleen ycrnpdndpq gpwcyttdpe krydycdile |
| 181 | ceeecmhcsg enydgkiskt msglecqawd sqsphahgyi pskfpnknlk knycrnpdre |
| 241 | lrpwcfttdp nkrwelcdip rcttpppssg ptyqclkgtg enyrgnvavt vsghtcqhws |
| 301 | aqtphthnrt penfpcknld enycrnpdgk rapwchttns qvrweyckip scdsspvste |
| 361 | qlaptappel tpvvqdcyhg dgqsyrgtss ttttgkkcqs wssmtphrhq ktpenypnag |
| 421 | ltmnycrnpd adkgpwcftt dpsvrweycn lkkcsgte [symbol] [symbol] [symbol] |
| 481 | [symbol] [symbol] [symbol] [symbol] [symbol] [symbol] [symbol] [symbol] |
| 541 | [symbol] [symbol] [symbol] aapsfdcgkp qvepkkcpgr vvggcvahph swpwqvslrt |
| 601 | rfgmhfcggt lispewvlta ahcleksprp ssykvilgah qevnlephvq eievsrlfle |
| 661 | ptrkdiallk lsspavitdk vipaclpspn yvvadrtecf itgwgetqgt fgagllkeaq |
| 721 | lpvienkvcn ryeflngrvq stelcaghla ggtdscqgds ggplvcfekd kyilqgvtsw |
| 781 | glgcarpnkp gvyvrvsrfv twiegvmrnn |

Note:
Kringle 4 region is in bold and underlined; Kringle 5 region is in bold
and italic; catalytic domain is underlined.

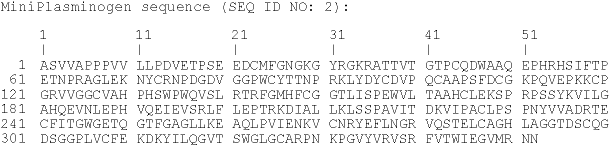

```
MicroPlasminogen sequence (SEQ ID NO: 3):
                10 20 30 40
ASMTGGQQMG RGSPGRVAAP SFDCGKPQVE PKKCPGRVVG
                50 60 70 80
GCVAHPHSWP WQVSLRTRFG MHFCGGTLIS PEWVLTAAHC
                90 100 110 120
LEKSPRPSSY KVILGAHQEV NLEPHVQEIE VSRLFLEPTR
              130 140 150 160
KDIALLKLSS PAVITDKVIP ACLPSPNYVV ADRTECFITG
              170 180 190 200
WGETQGTFGA GLLKEAQLPV IENKVCNRYE FLNGRVQSTE
              210 220 230 240
LCAGHLAGGT DSCQGDSGGP LVCFEKDKYI LQGVTSWGLG
              250 260
CARPNKPGVY VRVSRFVTWI EGVMRNN
```

### REFERENCES

1. U. Christensen, L. Sottrup-Jensen, S. Magnusson, T. E. PetersenI. Clemmensen, Enzymic properties of the neo-plasmin-Val-442 (miniplasmin), Biochim Biophys Acta 567 (1979):472-481.
2. K. A. Ney, S. V. Pizzo, Fibrinolysis and fibrinogenolysis by Val442-plasmin, Biochim Biophys Acta 708 (1982):218-224.
3. H. L Wu., G. Y. Shi, M. L. Bender, Preparation and purification of microplasmin, Proc Natl Acad Sci U.S.A. 84 (1987):8292-8295.
4. H. L Wu., G. Y. Shi, R. C. Wohl, M. L. Bender, Structure and formation of microplasmin, Proc Natl Acad Sci U.S.A. 84 (1987):8793-8795.
5. J. E. Wang, Reich, Structure and function of microplasminogen: II. Determinants of activation by urokinase and by the bacterial activator streptokinase, Protein Sci 4 (1995):1768-1779.
6. V. Novokhatny, K. Taylor, T. P. Zimmerman, Thrombolytic potency of acid-stabilized plasmin: superiority over tissue-type plasminogen activator in an in vitro model of catheter-assisted thrombolysis, J Thromb Haemost 1 (2003):1034-1041.
7. V. J. Marder, K. Landskroner, V. Novokhatny, T. P. Zimmerman, M. Kong, J. J. Kanouse, G. Jesmok, Plasmin induces local thrombolysis without causing hemorrhage: a comparison with tissue plasminogen activator in the rabbit, Thromb Haemost 86 (2001):739-745.
8. N. Nagai, E. Demarsin, B. Van Hoef, S. Wouters, D. Cingolani, Y. Laroche, D. Cohen, Recombinant human microplasmin: production and potential therapeutic properties, J Thromb Haemost 1 (2003):307-313.
9. F. W. Studier, Protein production by auto-induction in high density shaking cultures, Protein Expr Purif 41 (2005):207-234.
10. J. V. Jensen, Process of stabilizing therapeutically useful plasmin solutions. U.S. Pat. No. 3,950,513, Novo Terapeutisk Laboratorium A/S (DK), (1976).
11. E. Komorowicz, K. Kolev, R. Machovich, Fibrinolysis with des-kringle derivatives of plasmin and its modulation by plasma protease inhibitors, Biochemistry 37 (1998):9112-9118.
12. K. Kolev, E. Komorowicz, W. G. Owen, R. Machovich, Quantitative comparison of fibrin degradation with plasmin, miniplasmin, neurophil leukocyte elastase and cathepsin G, Thromb Haemost 75 (1996):140-146.
13. K. Kolev, E. Komorowicz, R. Machovich, Heparin modulation of the fibrinolytic activity of plasmin, miniplasmin and neutrophil leukocyte elastase in the presence of plasma protease inhibitors, Blood Coagul Fibrinolysis 5 (1994):905-911.
14. B. WIMAN and D. COLLEN, On the Kinetics of the Reaction between Human Antiplasmin and Plasmin, European Journal of Biochemistry 84 (1978):573-582
15. Y. Cao, A. Chen, S. S. An, R. W. Ji, D. Davidson, M. Llinas, Kringle 5 of plasminogen is a novel inhibitor of endothelial cell growth, J Biol Chem 272 (1997):22924-22928.
16. Y. Zhou, Q. Zheng, J. Gao, J. Gu, High level expression of kringle 5 fragment of plasminogen in Pichia pastoris, Biotechnol Lett 27 (2005):167-171.
17. U. Christensen, K. Bangert, S. Thorsen, Reaction of human alpha2-antiplasmin and plasmin stopped-flow fluorescence kinetics, FEBS Lett 387 (1996):58-62.
18. J. H. Graversen, C. Jacobsen, B. W. Sigurskjold, R. H. Lorentsen, S. K. Moestrup, H. C. Thogersen, M. Etzerodt, Mutational analysis of affinity and selectivity of kringle-tetranectin interaction. Grafting novel kringle affinity onto the trtranectin lectin scaffold, J Biol Chem 275 (2000):37390-37396.

What is claimed is:

1. A method for refolding a recombinant plasminogen polypeptide, comprising:
(a) solubilizing a plasminogen polypeptide in a solubilization buffer, said solubilization buffer comprising a high concentration of chaotroph, a reducing agent, redox reagents, and having a pH of about 9.0 to about 11.0, thereby producing a solubilized plasminogen polypeptide solution; and
(b) rapidly diluting said solubilized plasminogen polypeptide solution with a refolding buffer by adding said solubilized plasminogen polypeptide solution into the refolding buffer, thereby producing diluted solubilized plasminogen polypeptide solution, wherein the refolding buffer comprises arginine and has a pH of about 8.0 to about 10.0; and
(c) incubating the diluted solubilized plasminogen polypeptide solution, thereby producing a refolded plasminogen polypeptide.

2. The method of claim 1, wherein the chaotroph in the solubilization buffer is urea.

3. The method of claim 2, wherein said urea is at about 4 M to about 8 M concentration.

4. The method of claim 2, wherein the urea in the diluted solubilized plasminogen polypeptide solution is about 0.1 M to about 2 M concentration.

5. The method of claim 1, wherein the refolding buffer contains about 0.05 M to about 0.5 arginine.

6. The method of claim 1, wherein the refolding buffer has a pH of about 9.0 to about 9.5.

7. The method of claim 1, wherein said reducing agent in the solubilization buffer is β-mercaptoethanol, dithiothreitol (DTT), or both.

8. The method of claim 1, wherein said redox reagents are oxidized and reduced glutathione.

9. The method of claim 1, wherein the refolding buffer further comprises a reversible protease inhibitor.

10. The method of claim 1, wherein the solubilization buffer comprises about 100 mM Tris, about 8 M urea, about 1 mM glycine, about 1 mM EDTA, about 10 mM β-mercaptoethanol, about 10 mM DTT, about 1 mM reduced glutathion (GSH), and about 0.1 mM oxidized glutathion (GSSG), at pH between about 9.5 to about 10.5.

11. The method of claim 10, wherein the refolding buffer comprises about 20 mM Tris, about 0.2 M arginine, at pH about 9.0 to pH about 9.5.

12. The method of claim 11, wherein the protein concentration in said solubilized plasminogen polypeptide solution is adjusted to about 2 mg/ml before dilution.

13. The method of claim 12, wherein said solubilized plasminogen polypeptide is diluted into about twenty-fold refolding buffer.

14. The method of claim 1, wherein the plasminogen polypeptide is dissolved from bacteria inclusion bodies.

15. The method of claim 1, wherein the plasminogen polypeptide comprises the amino acid residues 561-810 of SEQ ID NO:1.

16. The method of claim 1, wherein the plasminogen polypeptide comprises the amino acid sequence of SEQ ID NO:2.

17. The method of claim 1, wherein the plasminogen polypeptide comprises the amino acid sequence of SEQ ID NO:3.

18. The method of claim 1, further comprises purifying said refolded plasminogen polypeptide.

19. The method of claim 1, wherein said refolded plasminogen polypeptide is purified by size exclusion chromatography (SEC).

20. The method of claim 1, wherein said refolded plasminogen polypeptide is purified by ion exchange chromatography (IEC).

21. The method of claim 1, wherein said refolded plasminogen polypeptide is purified by benzamidine affinity chromatography.

22. The method of claim 18, further comprising treating purified refolded plasminogen polypeptide by a plasminogen activator to generate biologically active plasmin polypeptide.

23. The method of claim 22, wherein the plasminogen polypeptide is dissolved from bacteria inclusion bodies.

24. A composition comprising a plasmin polypeptide produced by the method of claim 23.

25. The composition of claim 24, further comprising a pharmaceutically acceptable excipient.


SEQUENCE LISTING (5 sequences)

SEQ ID NO: 1 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 2 - protein, 352 aa, Homo sapiens
ASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 3 - protein, 267 aa, Artificial Sequence
  Synthetic Construct
ASMTGGQQMGRGSPGRVAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 4 - protein, 261 aa, Homo sapiens
KLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 5 - protein, 268 aa, Artificial Sequence
  Synthetic Construct
MASMTGGQQMGRGSPGRVAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN